(19) United States
(12) Patent Application Publication
(10) Pub. No.: US 2019/0256884 A1
(43) Pub. Date: Aug. 22, 2019

(54) METHODS OF PRODUCING A FERMENTATION PRODUCT IN TRICHODERMA

(71) Applicant: NOVOZYMES A/S, Bagsvaerd (DK)

(72) Inventors: Abigail Jang, Roseville, CA (US); Sandra Merino, Davis, CA (US); Kim Hansen, Vaerloese (DK); Glenn Munkvold, Ottawa (CA); Bogie Ploch, Ottawa (CA)

(73) Assignee: NOVOZYMES A/S, Bagsvaerd (DK)

(21) Appl. No.: 16/315,342

(22) PCT Filed: Jul. 7, 2017
(86) PCT No.: PCT/US2017/041113

§ 371 Date: Jan. 4, 2019

Related U.S. Application Data
(60) Provisional application No. 62/467,542, filed on Mar. 6, 2017.
(60) Provisional application No. 62/359,478, filed on Jul. 7, 2016.

(51) Int. Cl. C12P 21/02; C12N 15/66; C12N 15/52; C12N 9/24

(52) U.S. Cl. CPC C12P 21/02; C12N 15/66; C12Y 302/01026; C12N 9/2402; C12N 15/52

(57) ABSTRACT

This application discloses methods for fermenting recombinant Trichoderma reesei comprising a heterologous invertase gene, using sucrose as carbon source.

### REFERENCE TO SEQUENCE LISTING

This application contains a Sequence Listing in computer readable form, which is incorporated herein by reference.

### FIELD OF THE INVENTION

The present invention relates to a process of producing a fermentation product in a Trichoderma reesei cell in a fermentation medium comprising sucrose. The fermentation product may be a protein product, e.g., an enzyme product.

### BACKGROUND OF THE INVENTION

Trichoderma reesei is a well-known filamentous fungus that in recent years frequently has been used in fermentation processes, such as fermentation processes for the production of protein products, in particular for production of enzymes.

Trichoderma reesei is known to produce many cellulases and hemicellulases and the organism has frequently been used to produce enzyme products comprising cellulases and/or hemicellulases. The use of T. reesei, however, is not limited to production of cellulases and hemicellulases but also the production of other enzyme products.

Sucrose has typically been used as a carbon source for many microbial fermentation processes, including protein production in bacteria, e.g., Bacillus sp., and in filamentous fungi, such as Aspergillus sp. However, Trichoderma reesei strains do not utilize sucrose efficiently as a carbon source.

Sucrose has a beneficial high solubility in water, meaning that it can advantageously be used as high concentrated feed in fed-batch fermentations because the carbon source is delivered in adequate amounts without diluting the broth to much.

Further, molasses, a by-product from sugar production, contains high amounts of sucrose and therefore can be used as a relatively cheap carbon source for fermentation processes, in particular in locations close to sugar refineries.

Dernt et al., 2013, Biotechnology for Biofuels 6:62 disclose a mutation of the xylanase regulator 1 (xyr1) that causes a glucose blind hydrolase expressing phenotype in Trichoderma reesei, i.e., the strain does not sense the presence of glucose to affect gene expression. The mutation was identified as an alanine to valine substitution in position 824 of xyr1.

It would be desirable to use sucrose as a carbon source for T. reesei fermentations in order to benefit from this convenient nutrient.

### SUMMARY OF THE INVENTION

The invention provides a method of producing a fermentation product, comprising fermenting a recombinant Trichoderma reesei cell in a medium comprising sucrose.

### DEFINITIONS

Heterologous polypeptide: The term “heterologous polypeptide” means a polypeptide that is not naturally produced by Trichoderma reesei. The heterologous polypeptide may be derived from a different organism or it may be a variant, i.e., a polypeptide that differs from a naturally occurring polypeptide comprising a substitution, insertion or deletion. The term “heterologous polypeptide” includes fusion proteins, chimeric proteins, and variants.

Invertase: The term “invertase” means a polypeptide having invertase activity. Invertases (EC 3.2.1.26) catalyze the hydrolysis of sucrose into glucose and fructose. The systematic name for Invertase is β-D-fructofuranoside fructohydrolase, but the enzyme is also known under other names such as beta-fructofuranosidase, saccharase, sucrase and beta-fructosidase. Invertases are found in Glycoside Hydrolase Family 32 (GH-32) according to the Glycoside Hydrolase classification (Henrissat, 1991, Biochem. J. 280: 309-316 and cazy.org). An example of an invertase gene is a gene encoding an extracellular invertase from Aspergillus niger having the amino acid sequence of the mature protein of SEQ ID NO: 1. In some embodiments the mature protein of SEQ ID NO: 1 is amino acids 54 to 589 of SEQ ID NO: 1.

Native polypeptide: The term “native polypeptide” means a polypeptide that is naturally produced by Trichoderma reesei.

Operably linked: The term “operably linked” means a configuration in which a control sequence is placed at an appropriate position relative to the coding sequence of a polynucleotide such that the control sequence directs expression of the coding sequence.

Proteinaceous product: The term “proteinaceous product” means a product prepared by fermentation and comprising one or more polypeptide(s) of interest. The proteinaceous product may be a product comprising several different polypeptides of interest, e.g., a proteinaceous product for degrading cellulose may comprise at least one endoglucanase, at least one cellobiohydrolase and at least one beta-glucosidase. The proteinaceous product may in addition to one or more polypeptides of interest comprise further polypeptides, other components derived from the fermentation broth and components added during recovery and formulation of the product.

Recombinant: The term “recombinant” means that a Trichoderma reesei cell in which one or more genes encoding one or more polypeptides have been introduced.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

### DETAILED DESCRIPTION OF THE FIGURES

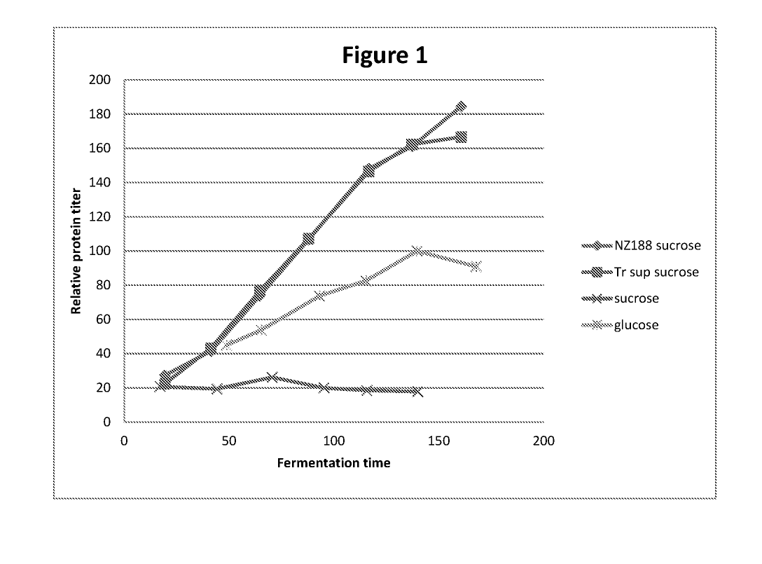

FIG. 1 shows the relative protein concentration produced by Trichoderma reesei cells as a function of fermentation time in the experiment described in Example 6.

### DETAILED DESCRIPTION OF THE INVENTION

The present invention relates to methods of producing a fermentation product, comprising fermenting a recombinant Trichoderma reesei cell in a fermentation medium comprising sucrose under conditions for producing a heterologous invertase and the fermentation product, wherein the recombinant Trichoderma reesei cell comprises one or more gene(s) encoding the heterologous invertase.

The present invention also relates to methods of producing a fermentation product, comprising fermenting a Trichoderma reesei cell in a fermentation medium comprising sucrose and a beta-glucosidase under conditions for producing the fermentation product and under conditions for formation of sophorose.

Trichoderma reesei Cells

T. reesei is a mesophilic filamentous fungus having the capacity to secrete large amounts of cellulolytic enzymes, and it has been used in the fermentation industry for many years for such a purpose. It is an anamorph of the ascomecetes Hypocrea jecorina and in this specification and claims all strains of Hypocrea jecorina and Trichoderma reesei are considered to be Trichoderma reesei strains regardless of the fact that some of the strains from a strictly taxonomical point should be considered as Hypocrea jecorina strains.

Any strain of T. reesei may be used according to the invention, however, it is preferred to use a T. reesei strain producing high amounts of extracellular enzymes such as strains based on QM6a, QM9414 and RutC30. These strains and a multitude of strains derived from these strains are all described in the art.

In some embodiments the T. reesei strain has a reduced catabolite repression system. Such a strain has the benefit that a promoter in which a wild-type strain is repressed in the presence of glucose will be less repressed in a strain having a reduced catabolite repression system compared with a wild-type strain. Fungal catabolite repression systems are known in the art and it is within the skill of the average practitioner in the field to identify suitable mutations leading to reduced catabolite repression and select a suitable T. reesei strain for the purpose of the present invention. One mutation leading to a reduced catabolite repression is a mutation in the crel gene (Strauss et al., 1995, FEBS Letters 376: 103-107). An example of a T. reesei strain having a crel mutation is Trichoderma reesei RutC30 that has also been extensively described in the literature, and this strain or strains derived from this strain will also be suitable for use according to the invention.

In some embodiments the T. reesei comprises a mutation in the xylanase regulator 1 (xyr1) that causes a glucose blind phenotype, such as a substitution of alanine to valine in position 824 (A824V) (Derntl et al., 2013, Biotechnology for Biofuels 6: 62 incorporated by reference). The A824V mutation in xyr1 is responsible for the strong deregulation of endo-xylanase expression and a highly elevated basal level of cellulase expression in T. reesei strains and is particularly beneficial if the recombinant T. reesei strain is used for producing native cellulases and/or hemicellulases or for producing heterologous proteins by use of T. reesei promoters derived from a cellulase or hemicellulase gene.

In other embodiments the recombinant T. reesei cell comprises both a mutation leading to reduced catabolite repression and a mutation in xyr1 that causes a glucose blind phenotype such as a T. reesei strain comprising a crel mutation and an A824V mutation in xyr1.

### Fermentation Products

The fermentation product may be a proteinaceous product, e.g., an enzyme. In some embodiments, the fermentation product is one or more enzymes selected from the group consisting of hydrolase, isomerase, ligase, lyase, oxidoreductase, and transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, lysozyme, mannosidase, mutanase, oxidase, pectinolytic enzyme, peroxidase, phytase, polyphenoloxidase, proteolytic enzyme, ribonuclease, transglutaminase, or xylanase. In particular, the fermentation product is one or more cellulases (cellobiohydrolase, endoglucanase, and/or beta-glucosidase) and/or one or more hemicellulases (acetylxylan esterase, arabinofuranosidase, feruloyl esterase, glucuronidase, xylanase, and/or xylosidase).

In some embodiments the proteinaceous product comprises only native polypeptides.

In other embodiments the proteinaceous product comprises heterologous polypeptides, optionally in addition to native polypeptides.

In some embodiments, the fermentation product is a whole broth product.

### Invertase

In some embodiments, the Trichoderma reesei cell comprises one or more genes encoding a heterologous polypeptide having invertase activity. The one or more genes may be any such genes encoding a heterologous polypeptide having invertase activity.

The invertase gene may be a bacterial or a fungal gene, where fungal genes are preferred. Examples of suitable invertase genes include invertase genes from Aspergillus niger, Aspergillus aculeatus, Aspergillus oryzae, Fusarium graminearum, Kluveromyces lactis, Penicillium chrysogenum, Penicillium hirsutum, Penicillium italicum, Saccharomyces cerevisiae, Talaromyces minoluteus, and Thielavia terrestris.

Preferred invertases include the invertase having the amino acid sequence of the mature protein of SEQ ID NO: 1, or having at least 80% sequence identity to SEQ ID NO: 1, e.g., at least 85% sequence identity, e.g., at least 90% sequence identity, e.g., at least 95% sequence identity, e.g., at least 96% sequence identity, e.g., at least 97% sequence identity, e.g., at least 98% sequence identity, e.g., at least 99% sequence identity to the mature protein of SEQ ID NO: 1. In an embodiment the mature protein of SEQ ID NO. 1 is the polypeptide consisting of amino acids 54-589 of SEQ ID NO: 1.

Other suitable invertases include the invertase from Aspergillus aculeatus (SEQ ID NO: 4), Penicillium hirsutum (SEQ ID NO: 5), Peniclllium italicum (SEQ ID NO: 6) and Talaromyces minioluteus (SEQ ID NO: 7), or any polypeptide having invertase activity and having at least 60% sequence identity, e.g., at least 70% sequence identity, e.g., at least 80% sequence identity, e.g., at least 85% sequence identity, e.g., at least 90% sequence identity, e.g., at least 95% sequence identity, e.g., at least 96% sequence identity, e.g., at least 97% sequence identity, e.g., at least 98% sequence identity, e.g., at least 99% sequence identity to any of these sequences.

Other invertases include the invertase having the amino acid sequence of the mature protein of SEQ ID NO: 1, and invertases that differ from the mature protein of SEQ ID NO: 1 by 1, 2, 3, 4, 5, 6, 7, 8, 9 or 10 alterations, e.g., substitutions, insertions, or deletions, preferably by 1, 2, 3, 4, 5, 6, 7, 8, 9 or 10 conservative substitutions.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

The invertase gene may be a natural gene or a non-natural gene, i.e., a gene where the amino acid and/or the nucleotide sequence has been altered in at least one position using recombinant DNA technologies.

The invertase gene should be operationally connected to a promoter, terminator and/or other regulatory elements necessary to direct expression of the gene in the T. reesei strain.

The invertase gene may be expressed using its own promoter, terminator and/or other regulatory elements, or it may be expressed using a heterologous promoter, terminator and/or other regulatory element. In this connection a heterologous promoter, terminator and/or other regulatory elements are understood as a promoter, terminator and/or other regulatory element that in nature is not found operationally connected to the gene.

The invertase gene may be inserted into the T. reesei strain using methods for transforming T. reesei as known in the art.

### Transformation of T. Reesei

The T. reesei strain may be transformed with one or more genes encoding one or more heterologous invertases and/or one or more genes encoding one or more polypeptides, and isolating a transformant comprising the one or more genes. Two or more copies of a gene encoding the one or more polypeptides, e.g., two, three, or four copies, may be introduced into the T. reesei strain. Techniques for transforming T. reesei are known in the art and the present invention is not limited in any way by the selected transformation technique. Suitable procedures for transformation of Trichoderma host cells are described in EP 238 023 and Yelton et al., 1984, Proceedings of the National Academy of Sciences USA 81: 1470-1474 and these references are incorporated in the present description by reference.

The one or more gene(s) should be operably linked to promoters, terminators and/or other regulatory elements capable of expressing the gene in T. reesei. The invention is not limited to any particular promoter, terminator and/or other regulatory elements but it is preferred to use promoters, terminators and/or other regulatory elements known to direct a high expression level in Trichoderma. This is all known in the art and it is completely within the skill of the average practitioner to select suitable promoters, terminators and/or other regulatory elements for use according to the present invention.

In a particular embodiment the promoter(s) directing expression of the one or more further gene(s) is/are subject to catabolite repression, such as promoters derived from genes encoding cellulases and hemicellulases; and the T. reesei cell comprises a mutation leading to reduced catabolite repression, such as a mutation in crel. In a further embodiment the promoter(s) directing expression of the one or more further gene(s) is/are derived from genes encoding cellulases and hemicellulases, and the T. reesei cell further comprises a xyr1 mutation which makes the cell glucose blind, such as an A824V substitution of xyr1.

### Fermentation Medium

The fermentation medium comprises sucrose, which is hydrolyzed into glucose and fructose, e.g., using a polypeptide having invertase activity or using an acid (e.g., acetic acid, citric acid, hydrochloric acid, phosphoric acid, or sulfuric acid)) at a pH of 1-3, e.g., pH 2.

In contrast to wild-type T. reesei cells which cannot utilize sucrose as a carbon source efficiently, recombinant T. reesei cells comprising one or more genes encoding an invertase grow well on sucrose, and can therefore use sucrose as a carbon source in the fermentation process.

Sucrose is an abundant source produced by extraction from certain crops, such as sugar beets and sugar cane. Thus, sucrose is readily available in many countries either as a pure refined product consisting of more than 99% sucrose or available in form of molasses, a by-product of the refining of sugarcane or sugar beets. Further, sucrose has the benefit of a high solubility in water meaning that a highly concentrated sucrose solution may be used as the feed in a fed-batch fermentation process whereby a high amount of available carbon source can be supplied to the fermentation without too high dilution of the fermentation broth with the water necessary to dissolve the carbon source in the feed. Thus, sucrose has several advantages in the fermentation industry and the present invention renders these benefits available for the fermentation of T. reesei strains.

In some embodiments, the fermentation medium comprises a beta-glucosidase. The beta-glucosidase may be added exogenously to the fermentation medium or may be produced by the Trichoderma reesei cell. The beta-glucosidase catalyzes the conversion of glucose to sophorose, which is an inducer for production of cellulolytic and hemicellulolytic enzymes.

### Fermentation

The invention is not limited in any way to the fermentation process performed but can be applied to any fermentation process, such as batch fermentation, fed-batch fermentation or continuous fermentation. The invention may even be applied to solid state fermentation where sucrose or a sucrose containing material is used in the fermentation.

The T. reesei cells are cultivated in a nutrient medium suitable for production of the polypeptide(s) using methods known in the art. For example, the cells may be cultivated by shake flask cultivation, or small-scale or large-scale fermentation (including continuous, batch, fed-batch, or solid state fermentations) in laboratory or industrial fermentors in a suitable medium comprising sucrose and under conditions allowing the polypeptide to be expressed and/or isolated. The cultivation takes place in a suitable nutrient medium comprising sucrose and optionally other carbon and further comprising nitrogen sources and inorganic salts, using procedures known in the art. Suitable media are available from commercial suppliers or may be prepared according to published compositions (e.g., in catalogues of the American Type Culture Collection). If the polypeptide of interest is secreted into the nutrient medium, the polypeptide can be recovered directly from the medium. If the polypeptide is not secreted, it can be recovered from cell lysates.

The fermentation product may be recovered using methods known in the art. For example, the product may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The proteinaceous product may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide forms the proteinaceous product.

### Fermentation Broth Formulations or Cell Compositions

The present invention also relates to a fermentation broth formulation or a cell composition comprising a polypeptide of interest. The fermentation broth product further comprises additional ingredients used in the fermentation process, such as, for example, cells, cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the T. reesei cells are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### EXAMPLES

### Materials and Methods

### Growth Media:

| Fermentation medium | | |
|---|---|---|
| Soja meal | 40 | g/liter |
| MgSO₄,7H₂O | 8 | g/liter |
| K₂SO₄ | 9 | g/liter |
| Citric acid | 1 | g/liter |
| K₂HPO₄ | 3 | g/liter |
| (NH₄)₂SO₄ | 8 | g/liter |
| ZnSO₄,7H₂O | 0.081 | g/liter |
| CuSO₄,5H₂O | 0.039 | g/liter |
| FeSO₄,7H₂O | 0.384 | g/liter |
| MnSO₄,H₂O | 0.096 | g/liter |
| CaCO₃ | 3 | g/liter |
| Sucrose or glucose | 12 | g/liter |
| H₃PO₄ 85% w/w | 4 | ml/liter |
| Defoaming agent | 1 | ml/liter |
| Seed medium NNCell1: | | |
| Glycerol | 20 | g/liter |
| Soy grits | 10 | g/liter |
| (NH₄)₂SO₄ | 1.5 | g/liter |
| K₂HPO₄ | 2 | g/liter |
| MgSO₄,7H₂O | 0.4 | g/liter |
| Trace metals | 0.2 | ml/L |
| CaCO₃ | 2.5 | g/liter |

SOC medium
20 g/liter Tryptone
5 g/liter Yeast extract
4.8 g/liter MgSO₄
3.603 g/liter dextrose
0.5 g/liter NaCl
0.186 g/liter KCl
2XYT plates
16 g/liter Tryptone
10 g/liter Yeast extract
5 g/liter NaCl
15 g/liter Agar
COVE plates
342.3 g Sucrose
20 ml Cove Salt Solution
10 ml 1M Acetamide
10 ml 1.5M CsCl
25 g Agar Noble
Water to 1 liter
COVE Salt Solution
26 g KCl
26 g MgSO₄ 7H₂O
76 g KH₂PO₄
50 ml COVE Trace Elements
Water to 1 liter
COVE Trace Elements
0.04 g Nz₂B₄O₇ 10H₂O
0.4 g CuSO₄ 5H₂O
1.2 g FeSO₄ 7H₂O
0.7 g MnSO₄ H₂O
0.8 g Na₂MoO₂ 2H₂O
10 g ZnSo₄ 7H₂O
Water to 1 liter
COVE2 plates
30 g Sucrose
20 ml Cove Salt Solution
10 ml 1M Acetamide
25 g Agar Noble
Water to 1 liter
Trichoderma Minimal plates
10 ml Cove Salt Solution
0.3 g CaCl₂, 2H₂O
3 g (NH₄)₂SO₄
12.5 g Agar Noble
Water to 480 ml

The mixture was autoclaved for 30 minutes at 121° C. and cooled to approximately 50° C. and the required amount of glucose or sucrose was added as a 50% aqueous solution to a final concentration of 2% glucose or sucrose.

### DNA Manipulation

Enzymes for DNA manipulation such as restriction enzymes were provided from Clontech Laboratories, Inc. Mountain View, Calif., USA and used according to the manufacturer's instructions.

### Fermentation

The fermenters used in the Examples were standard lab scale (2 liter) fermenters.

Brown sugar was prepared as described in WO 2012/104176, Example 1.

### Analysis

Total protein was measured using a Pierce™ BCA Protein Assay Kit (ThermoFisher Scientific cat. no. 23227, provided by Life Technologies Europe BV; Naerum, Denmark) according to the manufacturer's instructions.

### Example 1

### Cloning and Preparing Plasmid Encoding the Aspergillus niger Suc1 Gene

The expression vector pMJ09 (WO 2005/067531) was used as basis for the expression vector for this Example.

The Aspergillus niger suc1 gene encoding an invertase was PCR amplified from genomic DNA prepared fromAspergillus niger ATCC 1015, using the PCR primers shown below:

Suc1 F vector flk
(SEQ ID NO: 2)
attacgaattgtttaaacgtgctttacttcactcgtgcatgggg
Suc1 R vector flk
(SEQ ID NO: 3)
aaatggattgattgtctcaccacgtgcacattcatattccgc

underlined bases correspond to the gene sequence of Suc1, whereas bases not underlined correspond to vector sequence.

### Reaction Mixture

| | | |
|---|---|---|
| 5 × Phusion HF buffer | 10 | μl |
| dNTPs (10 mM each) | 1.5 | μl |
| Primers (50 μM) | 1 | μl each |
| Genomic DNA (10 ng/μl) | 10 | μl |
| Water | to 50 | μl |
| Phusion polymerase (2 U/μl) | 0.5 | μl |

PCR conditions:

| | |
|---|---|
| Step 1 | 98° C. for 30 seconds |
| Step 2 | 98° C. for 10 seconds |
| Step 3 | 56° C. for 15 seconds |
| Step 4 | 72° C. for 160 seconds |

Steps 2-4 were repeated for 34 cycles whereafter the reaction mixtures were kept on hold at 10° C.

Five μl of the reaction mixture was electrophoresed on a 0.8% agarose gel using TAE buffer and a fragment of the expected size (3886 bp) was observed.

Plamid pMJ09 was digested with the restriction endonuclease Accl and purified. The purified linearized vector and the purified PCR amplified suc1 gene were assembled and inserted into E. coli using the Clontech Infusion cloning protocol and electro-transformed into Top10 electrocompetent E. coli cells (Clontech Laboratories, Inc, Mountain View, Calif., USA).

Transformed cells were resuspended in 1 ml of SOC medium and 20 μl and 200 μl of the transformed cells were spread onto 2XYT plates containing 100 mg/ml ampicillin and incubated at 37° C. until the next day where transformed colonies had emerged.

Eight colonies were selected and grown overnight whereafter plasmid DNA was prepared from each culture. The plasmid preparations were digested with the restriction endonuclease Accl, where transformants with the suc1 gene inserted into the vector would yield three restriction fragments (925, 2000 and 6685 base pairs), whereas vectors without insert would yield two fragments (1528 and 5683 base pairs).

Two transformants with the correct restriction fragment pattern were selected. The plasmids from these transformants were sequenced and one transformant was confirmed to contain the suc1 gene without any mutation. The plasmid from this transformant was named pVCK12TRI001.

### Example 2

### Transforming T. reesei with pVCK12TRI001 Comprising the Aspergillus niger suc1 Gene

Plasmid pVCK12TRI001 was linearized with the restriction endonuclease Pmel and transformed into the Trichoderma reesei RutC30 strain essentially as described in WO 2008/151079, Example 6 and the transformation was spread onto COVE plates.

Twenty-one transformants were selected and transferred to COVE2+10 mM uridine plates and incubated at 28° C. for 22-26 days.

The transformants were subcultured onto new COVE2 plates, Trichoderma minimal plates+2% sucrose, and Trichoderma minimal plates+2% glucose and incubated 28° C. for how 8 days. All transformants grew well on Trichoderma minimal plates+2% sucrose, Trichoderma minimal plates+2% glucose, and COVE2 plates. The untransformed Trichoderma reesei RutC30 strain did not grow on Trichoderma minimal plates+2% sucrose but grew as expected on Trichoderma minimal plates+2% glucose.

### Example 3

### Fermentation of the T. reesei RutC30 Strain in a Fermentation Medium Comprising Sucrose

Three fermenters were each filled with 1.1 kg fermentation medium and sterilized by heating for one hour at 123° C. After cooling to 25° C., the pH was adjusted to 5.0 using H₃PO₄ and/or ammonium hydroxide. The fermenters were inoculated with a shake flask with a preculture of the T. reesei RutC30 mutant strain.

After 18 hours, the additional carbon source shown in Table 1 was fed to the three fermenters. The fermenters were maintained at an oxygen saturation level of approximately 40%. The fermentations ran for 6 days.

TABLE 1

| Fermenter | Carbon source in feed |
|---|---|
| 1 | Brown sugar |
| 2 | 52% sucrose |
| 3 | 52% sucrose + Brown sugar (9:1) |

Biomass and CO₂ production were measured. Sucrose dosing yielded very low CO₂ production and biomass formation. When portions of the sucrose were replaced by brown sugar, CO₂ production and biomass formation were increased but were still lower than if only brown sugar was dosed.

Sucrose dosing yielded very low protein and cellulase production. When a portion of the sucrose was replaced with brown sugar, higher protein and cellulase production were achieved but the level was far below what was obtained using brown sugar alone. These results show that sucrose is a poor carbon source for cellulase production by T. reesei.

### Example 4

### Fermentation of Recombinant T. reesei (xyr1) Comprising the A. niger suc1 Gene

A recombinant T. reesei RutC30 mutant having the A. niger suc1 gene and an A824V substitution in the xylanase regulator 1 (xyr1) gene causing a “glucose blind” phenotype was prepared according to the method described in Example 2.

Three fermenters were prepared and performed as described in Example 3 and inoculated with the recombinant T. reesei mutant. The fermentations ran for 8 days. After 18 hours, the carbon source as shown in Table 2 was fed to the three fermenters using a feed rate beginning at 1 g/hour, increasing to 10 g/hour after 25 hours of feeding, and then decreasing to 4.5 g/hour after 162 hours of feeding. Because the oxygen level dropped to 0, the feed rate for fermenter 2 was reduced by 80% after 89 hours and the feed was stopped from 96 to 136 hours, and the feed rate for fermenter 3 was reduced by 80% from 89-101 hours and then increased to 50% of the original level. Samples for total protein were collected at the end of the fermentation.

TABLE 2

| Fermenter | Carbon source in feed |
|---|---|
| 1 | Brown sugar |
| 2 | 52% sucrose |
| 3 | 52% sucrose + brown sugar (9:1) |

The biomass yields for fermenters 2 and 3 were very high (approximately 100 g dry weight/kg culture broth) whereas in fermenter 1 the biomass yield was 40 g dry weight/kg culture broth.

Total extracellular protein was high for fermenter 1, low for fermenter 2 and intermediate for fermenter 3.

The results demonstrate that sucrose is a very good carbon source for the recombinant T. reesei mutant comprising a suc1 gene, and generates a high yield of biomass.

In order to obtain high extracellular protein production, inclusion of an inducer, such as brown sugar, in the feed is required.

### Example 5

### Fermentation of Recombinant T. reesei Mutant (xyr1) Comprising the A. niger Suc1 Gene Using Adjusted Feed Rate

Fermentation 3 of Example 4 (52% sucrose+brown sugar (9:1)) was repeated with a lower feed rate to avoid an unacceptably low oxygen level. A feed rate of approximately 50% of the feed rate from Example 4 was used, i.e., a feed rate starting at 1 g/hour, increasing to 5 g/hour after 25 hours of feeding and then decreasing to 2.5 g/hours after 162 hours of feeding.

The biomass yield was 40 g dry weight/kg culture broth, which is similar to the biomass yield obtained in Example 4, fermentation 1, and lower than the biomass yields obtained in Example 4, fermentations 2 and 3. The extracellular protein yield obtained was slightly lower than the protein yield in Example 4, fermentation 1, but higher than the protein yields in Example 4, fermentations 2 and 3 despite the reduced amount of feed added.

### Example 6

### Preparation of Fermentation Media:

Glucose medium was prepared by dissolving glucose monohydrate in tap water to a concentration of 55% w/w glucose and sterilizing by autoclaving at 121° C. for 60 minutes.

Sucrose medium was prepared by dissolving sucrose in tap water to a concentration of 52% w/w sucrose and sterilizing by autoclaving at 121° C. for 60 minutes.

60% w/w BG-sucrose medium was prepared by dissolving 3900 g of sucrose and 10.5 g of citric acid in 5 liters of tap water. This solution was heated to >95° C. for 30 minutes (to hydrolyze sucrose) and then cooled to <50° C. The pH was adjusted to 4.5 using NaOH and the solution was split into two portions of 2.5 liters. Twenty-five ml of Novozym 188 (commercial beta-glucosidase product from Novozymes A/S) were added to the first portion (“Novozym 188 sucrose” in Table 3). Five ml of filter sterilized supernatant from the fermentation of a recombinant Trichoderma reesei strain expressing beta-glucosidase, cellobiohydrolase and endoglucanase were added to the second portion (“T. reesei sup sucrose” in Table 3). Each of the portions was incubated at 50° C. for 4 days and sterilized by autoclaving at 121° C. for 60 minutes.

### Fermentation Experiments:

Spores of a Trichoderma reesei strain were inoculated into 500 ml shake flasks containing 200 ml of NNCell-1 medium and incubated with shaking at 250 rpm for 2 days at 26° C. The seed culture broth was transferred to 2 liter fermenters containing a medium containing soy meal, sucrose and salts. Fermentations were run at 28° C., pH 4.5-4.8 (controlled using phosphoric acid and ammonium hydroxide), and an aeration of 0.75-1.5 L/min. When the carbon dioxide level started to drop (indicating that the sucrose in the main medium had been consumed) feeding was started using the different media described in the “Preparation of fermentation media” section. The feeding rate was increased from 1 to 10 g/hour over the first 25 hours and then reduced gradually to maintain a dissolved oxygen level of 10-40% to make sure the carbon source was the limiting component in the cultures throughout the fermentation. The fermentations were terminated after 6-7 days. Extracellular protein concentration (used as an indicator for cellulase expression) was measured throughout the fermentations using the BCA assay. Maximum protein concentration for the fermentations relative to the glucose dosed fermentation is provided in Table 3 and the relative protein concentration as a function of fermentation time is shown in FIG. 1.

TABLE 3

| | Relative | |
|---|---|---|
| Dosing | max protein titer | Fermentation time at max titer |
| Novozym 188 sucrose | 184% | 161 hours |
| T. reesei sup sucrose | 167% | 161 hours |
| Sucrose | 26% | 71 hours |
| Glucose | 100% | 140 hours |

FIG. 1 shows that treating sucrose with a beta-glucosidase improved the fermentation yield of extracellular protein 6-7-fold greater than the yield obtained with sucrose and 1.7-1.8-fold greater than the yield obtained with glucose. The main reasons for this improvement are that the monosaccharides constituting sucrose (i.e., fructose and glucose) become available to the Trichoderma reesei due to hydrolysis by citric acid and high temperature, and that a disaccharide of glucose, which is formed by the action of the beta-glucosidase, acts as an inducer for enzyme production.

1. A method of producing a fermentation product, comprising fermenting a recombinant Trichoderma reesei cell in a fermentation medium comprising sucrose under conditions for producing a heterologous invertase and the fermentation product, wherein the recombinant Trichoderma reesei cell comprises one or more gene(s) each encoding the heterologous invertase.

2. The method of claim 1, wherein each of the one or more gene(s) encoding a heterologous invertase is derived from one or more microorganism(s).

3. The method of claim 2, wherein at least one of the one or more gene(s) encoding a heterologous invertase is a fungal gene.

4. The method of claim 3, wherein each of the one or more genes encoding a heterologous invertase has at least 60% sequence identity to SEQ ID NO: 1.

5. The method of claim 4, wherein each of the one or more genes encoding a heterologous invertase has the sequence of SEQ ID NO: 1, or differs from SEQ ID NO: 1 by one or several substitutions, including by one or several conservative substitutions.

6. A method of producing a fermentation product, comprising fermenting a Trichoderma reesei cell in a fermentation medium comprising sucrose and a beta-glucosidase, under conditions for producing the fermentation product and under conditions for formation of sophorose.

7. The method of claim 6, wherein the sucrose is hydrolyzed to fructose and glucose with an acid, including acetic acid, citric acid, hydrochloric acid, phosphoric acid, or sulfuric acid at a pH of 1-3.

8. The method of claim 6, wherein the sucrose is hydrolyzed to fructose and glucose by an invertase.

9. The method of claim 8, wherein the invertase is added exogenously to the fermentation medium.

10. The method of claim 8, wherein the invertase is produced recombinantly by the Trichoderma reesei cell.

11. The method of claim 6, wherein the beta-glucosidase is added exogenously to the fermentation medium.

12. The method of claim 6, wherein the beta-glucosidase is produced recombinantly by the Trichoderma reesei cell.

13. The method of claim 6, wherein the Trichoderma reesei cell is recombinant and has a mutation that provides reduced catabolite response compared with a corresponding Trichoderma reesei cell not having such a mutation.

14. The method of claim 13, wherein the mutation that provides reduced catabolite response is a mutation in a crel gene.

15. The method of claim 13, wherein the recombinant Trichoderma reesei cell further comprises a mutation in the xyr1 locus, the mutation resulting in the recombinant Trichoderma reeseicell becoming glucose blind.

16. The method of claim 15, wherein the mutation in the xyr1 locus is a substitution of alanine to valine in xyr1 at position 824 (A824V).

17. The method of claim 6, wherein the fermentation product is a proteinaceous product comprising one or more polypeptides.

18. The method of claim 17, wherein the one or more polypeptides are native to the Trichoderma reesei cell.

19. The method of claim 17, wherein the one or more polypeptides are heterologous to the Trichoderma reesei cell.

20. The method of claim 17, wherein one or more polypeptides are native and one or more polypeptides are heterologous to the Trichoderma reesei cell.

21. The method of claim 17, wherein the Trichoderma reesei cell comprises two or more copies of one or more genes encoding the one or more polypeptides.

22. The method of claim 17, wherein the one or more polypeptides are one or more enzymes.

23-29. (canceled)


SEQUENCE LISTING (7 sequences)

SEQ ID NO: 1 - protein, 628 aa, Aspergillus niger
MKLQTASVLLGSAAAASPSMQTRASVVIDYNVAPPNLSTLPNGSLFETWRPRAHVLPPNGQIGDPCLHYTDPSTGLFHVGFLHDGSGISSATTDDLATYKDLNQGNQVIVPGGINDPVAVFDGSVIPSGINGLPTLLYTSVSFLPIHWSIPYTRGSETQSLAVSSDGGSNFTKLDQGPVIPGPPFAYNVTAFRDPYVFQNPTLDSLLHSKNNTWYTVISGGLHGKGPAQFLYRQYDPDFQYWEFLGQWWHEPTNSTWGNGTWAGRWAFNFETGNVFSLDEYGYNPHGQIFSTIGTEGSDQPVVPQLTSIHDMLWVSGNVSRNGSVSFTPNMAGFLDWGFSSYAAAGKVLPSTSLPSTKSGAPDRFISYVWLSGDLFEQAEGFPTNQQNWTGTLLLPRELRVLYIPNVVDNALARESGASWQVVSSDSSAGTVELQTLGISIARETKAALLSGTSFTESDRTLNSSGVVPFKRSPSEKFFVLSAQLSFPASARGSGLKSGFQILSSELESTTVYYQFSNESIIVDRSNTSAAARTTDGIDSSAEAGKLRLFDVLNGGEQAIETLDLTLVVDNSVLEIYANGRFALSTWVRSWYANSTNISFFQNGVGGVAFSNVTVSEGLYDAWPDRQS

SEQ ID NO: 2 - DNA, 44 bp, Artificial sequence
  Synthetic construct
attacgaattgtttaaacgtgctttacttcactcgtgcatgggg

SEQ ID NO: 3 - DNA, 42 bp, Artificial sequence
  Synthetic construct
aaatggattgattgtctcaccacgtgcacattcatattccgc

SEQ ID NO: 4 - protein, 654 aa, Aspergillus aculeatus
MKLPTTTLALATGAAAAAASYHLDTTAPPPTNLSTLPNNTLFHLWRPRAHILPAEGQIGDPCAHYTDPSTGLFHVGFLHDGDGIAGATTANLATYTDTSGNGSFLIQPGGKNDPVAVFDGAVIPVGVNNTPTLLYTSVSFLPIHWSIPYTRGSETQSLAVARDGGRRFEKLDQGPVIADHPFAVDVTAFRDPFVFRSARLDVLLSLNEEVAGNETAVQQAVDGWTEKNAPWYVAVSGGVHGVGPAQFLYRQNGGNGSEFQYWEYLGEWWQEATNSSWGEEGTWAGRWGFNFETGNVLFLTEEGHDPQTGEVFVTLGTEGSGLPIVPQVSSIHDMLWAAGEVGVGSEEEGAKVEFSPSMAGFLDWGFSAYAAAGKVLPASSAVSKTSGVDVDRYVSFVWLTGDQYEQADGFPTAQQGWTGSLLLPRELKVQTVENVVDNELVREEGVSWVVGESNNKTATLRTLGITIARETKAALLANGSVTTEEARTLQTAAVVPFAQSPGSKFFVLTAQLEFPASARSSNLQSGFEILASELERTTVYYQFSNESLVVDRSQTSAAAPTNPGLDSFTESGKLRLFDVVENGQEKVETLDLTVVVDNSVVEVYANGRFALSTWARSWYDNSTQIRFFHNGEGEVQFKNVSVSEGLYNAWPERN

SEQ ID NO: 5 - protein, 621 aa, Penicillium hirsetum
MKLSSVMTVGLANRVLAANPHASPIDYNSAPPNLSTLANETLFDTWRPKAHLLPPSGKIGDPCGHYTDPKTGLFHVGWLHDGIAGATTDDLATYKDLNPDGAPVIIAGGKNDPLAVFDGSVIPSGINGLPTLLYTSVSYLPIHWSIPYTRGSETQSLAVSHDGGRNFTKLNQGPVIPTPPFAVNVTAFRDPFAFQSSILDKSVNSTKGTWYVAISGGVHDVGPTQFLYRQHDADFQYWEYLGQWWNEPVNTTWGKGDWAGGWGFNFEVGNVFGLNAEGYSEDGEIFITLGTEGSGLPIVPQVSSIHDMLWATGNVTNDDSVTFKPTMAGVLDWGLSAYAAAGKVLPASSQASEKSGAPDRFISYVWLTGDLFELAKGFPTAQQNWTGTLLLPRELNIRTIPNVVDNELSRESLTSWRVAREGSGEVDLETMGISIARETYSALTSGPSFVESGKTLSKAGSVPFNTSPSTKFFALTANISFPASARDSGIQAGFQVLSSGLESTTIYYQFSNESIIIDRSNTSAASKTTNGILSDNEAGRLRLFDVLENGKEKVETLELTVVVDNGVLEVYANGRFALGTWARSWYANSTEIKFFHNGVGEATFGNVTIFEGLYDAWPQRK

SEQ ID NO: 6 - protein, 687 aa, Penicillioum italicum
MKATTAASVLALVWEGATAQDYNAAPPDLATLPELSLFETWRPHTHVLPPNGQIGDPCAHYVDPETGRFHVGFLHNGTGIAAVLTEDLVHYYDVRPNGNYSIVAGGPNDPVAVFDGSVIPSGVDGKPTLLYTSVSSLPIHWTLAYTRGSESQSLAVTYDGEKNFTKLDIPPVIPVPPEGVNVTGFRDPYVFQNAALDQSIGNAEGTWYATISGGVHEVGPGMFLYQNTSPDFEHWEYLGEWYHEPANSTWGNGDWSKVFGYNFETTNFFGLTRQGYSYSGSPFFTIAVESSYVPIQPSVSSLHAQLWAAGSVGVPEGGNVTFTPDMVGVFDWGLASYAAAGKVVPASSQASTASGAPDRFISYVWLTGDVFGGVVGFPAAQQGWQNTLLTSRELYIKAIPNVVNNDLVKETGSWRLAADSDSTRNCVELETLGIDIARETYDAMTAAPSFSEPDKTLTSAGVIPFTRSPKTKFFILEADISFDARASDLQTGFQILASEFESTTIYYQFSNESIMIDRSKTSAAAGTTTGIDASPESGRLRLFDINDSCGNNGGNNGGHGGKGVYGGHSGQGGYAGHGGHDCGEKDGQGEHHSTNCKPDDKSGSNAHASRSSEVGGEHIETLSLTVVVDNSVLEVYANSRFVLSTWVRPWYQNSTEIRFFQNGEGKASFRNIRVADGLYDAYPDRDQ

SEQ ID NO: 7 - protein, 649 aa, Talaromyces minioluteus
MRSWCAGTAFAVALAVGVSAQNYNEPPPDLSTLPTGSLFETWRPKIHVLPPDGVVGDPCGHYDDPETGLFHVGFLHNGTGMAAVETADLVFYYDVNENGNYTIVPGGPNDPVAVFDGSVIPSGIDNKPTLLYTSVSSLPIHWTLPYTRGSESQSLAVTYDGGHNFSKLAIPPVIPEPPSGLNVTAFRDPYVFQNGILDTTLESVQGTWYATISGGQHDVGPGIFLYRNQGEDFEDWEYLGEWWQEPANSTWGNGDWAKLFGYNFETGNVFSLDEEGYNLDGDIFLTFGVEGAYAPIQAGVTSMHAMLWAAGNVSKADDGNVTFLPNMVGVLDWGLSSYAAAGKVLPHTSQASSNSGAPDRFISYIWLTGNDFGADADFPDAQQEWQHTLLLPRELSVETIHNVVNNELVQETGSWRVSESSGSSATHGSISGGDQNCVQLETLGIDIARETYEAITSASNSFSESGRILNSSSIIPFERSPTTRFFALRAQVSFQQSARQSALQSGFQILSSDLESTTVYYQFSNESIVIDRSNTSAAAQTTSGIDSSSESGRLRLFDVQEHCNQTYDGDIGDHGHEDTKVETLDLTIVVDNSVLEVFANSRFGVSTWARPWYANSTEIRFFQNGDGEVTFSNIAVYDGLYDAYPDRDR